(19) United States
(12) Patent Application Publication
(10) Pub. No.: US 2007/0003557 A1
(43) Pub. Date: Jan. 4, 2007

(54) Method for treating cancer using premedication

(76) Inventors: Andres Forero, Birmingham, AL (US); Shigeru Kobayashi, Princeton, NJ (US); Masami Tamaoka, Kamakura-shi (JP); Shose Taoka, Ichikawa-shi (JP)

Correspondence Address: NIXON & VANDERHYE, PC, 901 NORTH GLEBE ROAD, 11TH FLOOR, ARLINGTON, VA, 22203, US

(21) Appl. No.: 11/110,951

(22) Filed: Apr. 21, 2005

(51) Int. Cl. A61K 39/395; A61K 31/573; A61K 31/137

(52) U.S. Cl. 424/155.1; 514/171; 514/649

(57) ABSTRACT

The present invention relates to a method for treating cancer which comprises; administering to a patient in need thereof a therapeutically effective amount of at least two compounds selected from antihistamic agent, antiallergic agent and steroid, and then administering to the patient in need thereof a therapeutically effective amount of a humanized antibody or a human antibody which specifically binds to ganglioside GD3 or the antibody fragment thereof.

### FIELD OF THE INVENTION

A method for treating cancer which comprises; administering to a patient in need thereof a therapeutically effective amount of at least two compounds selected from antihistamic agent, antiallergic agent and steroid, and administering to the patient in need thereof a therapeutically effective amount of a humanized antibody or a human antibody which specifically binds to ganglioside GD3 or the antibody fragment thereof.

The present invention relates to a method for suppressing infusion reaction which is caused by administering to a patient in need thereof a therapeutically effective amount of a humanized antibody or a human antibody which specifically binds to ganglioside GD3 or the antibody fragment thereof which comprises; administering to a patient in need thereof a therapeutically effective amount of at least two compounds selected from antihistamic agent, antiallergic agent and steroid,

before administering to the patient in need thereof a therapeutically effective amount of a humanized antibody or a human antibody which specifically binds to ganglioside GD3 or the antibody fragment thereof.

### BACKGROUND OF THE INVENTION

In order to achieve effective clinical applications of an anti-GD3 antibody to human, an anti-GD3 human chimeric antibody was produced based on an anti-GD3 mouse antibody (U.S. Pat. No. 5,750,078, U.S. Pat. No. 5,866,692, U.S. Pat. No. 5,807,548, U.S. Pat. No. 6,437,098, U.S. Pat. No. 6,495,666). It is expected that, in comparison with the anti-GD3 mouse antibody, the anti-GD3 chimeric antibody reduces immunogenicity in human, prolongs half-life in blood and has a stronger anti-tumor effect. However, it was found as a result of clinical tests on other anti-GD3 chimeric antibodies in human that allergic reactions are induced due to induction of a human antibody against a V region derived from a mouse antibody in the anti-GD3 chimeric antibodies (Cancer J. From Scientific American, 3, S121 (1997)).

There has been known that administration of other antibodies also induces infusion reactions.

For example, when Rituxan, which is known as anti-CD20 human chimeric antibody, was administered to a patient, transient hypotension has occured. Premedication consisting of acetaminophen and diphenhydramine before infusion of Rituxan is known [Oncology, vol. 12, No. 12, 1998; Drugs 1999 July 58 (1) 79-88; Journal of Clinical Oncology, Vol 16, No 8 (August), 1998: pp 2825-2833; Blood, Vol 92, No 6 (Sep. 15), 1998: pp 1927-1932; Am J Health-Syst Pharm Vol 58 Feb. 1, 2001; Journal of Clinical Oncology, Vol 17, No 1 (January), 1999: pp 268-276].

As an example of the premedication in Campath-1H, diphenhydramine and acetaminophen are administered prior to Campath-1H infusion is known [Expert Rev. Anticancer Ther. 2 (1), 23-35 (2002); Medical Oncology, vol. 19, (Supplement), S49-S55, 2002; BLOOD, 15 May 2002, vol. 99, Number 10; Curr Opin Oncol 2000, 12: 574-581; Medical Oncology, vol. 18, no. 2, 99-107, 2001; CANCER PRACTICE July/August 2001, Vol. 9, No. 4].

### SUMMARY OF THE INVENTION

The present invention relates to a method for treating cancer which comprises;

administering to a patient in need thereof a therapeutically effective amount of at least two compounds selected from antihistamic agent, antiallergic agent and steroid,

and then administering to the patient in need thereof a therapeutically effective amount of a humanized antibody for a human antibody which specifically binds to ganglioside GD3 or the antibody fragment thereof.

The present invention relates the method for treating cancer which comprises administering to a patient in need thereof a therapeutically effective amount of a humanized antibody or a human antibody which specifically binds to ganglioside GD3 or the antibody fragment thereof,

wherein a therapeutically effective amount of at least two compounds selected from antihistamic agent, antiallergic agent and steroid are administered, before administering a therapeutically effective amount of the humanized antibody or the human antibody which specifically binds to ganglioside GD3 or the antibody fragment thereof.

Furthermore, the present invention relates to a method for suppressing infusion reaction which is caused by administering to a patient in need thereof a therapeutically effective amount of a humanized antibody or a human antibody which specifically binds to ganglioside GD3 or the antibody fragment thereof which comprises; administering to a patient in need thereof a therapeutically effective amount of at least two compounds selected from antihistamic agent, antiallergic agent and steroid,

before administering to the patient in need thereof a therapeutically effective amount of a humanized antibody or a human antibody which specifically binds to ganglioside GD3 or the antibody fragment thereof containing pharmaceutically acceptable composition.

### DETAILED DESCRIPTION OF THE INVENTION

The present invention relates to the following to (1) to (14).

(1) A method for treating cancer which comprises;

administering to a patient in need thereof a therapeutically effective amount of at least two compounds selected from antihistamic agent, antiallergic agent and steroid,

and then administering to the patient in need thereof a therapeutically effective amount of a humanized antibody or a human antibody which specifically binds to ganglioside GD3 or the antibody fragment thereof.

(2) The method according to (1), wherein the humanized antibody is a human chimeric antibody or a human complementary determining region (CDR)-grafted antibody.

(3) The method according to (1), wherein the humanized antibody or human antibody comprises:

CDR1, CDR2 and CDR3 of the H chain V region having the amino acid sequences represented by SEQ ID NOs: 5, 6 and 7, respectively; and

CDR1, CDR2 and CDR3 of the L chain V region having the amino acid sequences represented by SEQ ID NOs: 8, 9 and 10, respectively.

(4) The method according to (2), wherein the CDR-grafted antibody comprises:

the H chain V region of the antibody comprising the amino acid sequence represented by SEQ ID NO:1; and

the L chain V region of the antibody comprising the amino acid sequence represented by SEQ ID NO:2.

(5) The method according to (2), wherein the human chimeric antibody comprises:

the H chain V region of the antibody comprising the amino acid sequence represented by SEQ ID NO:3; and

the L chain V region of the antibody comprising the amino acid sequence represented by SEQ ID NO:4.

(6) The method according to (1), wherein the antihistamic agent is H1 blocker or H2 blocker.

(7) The method according to (1), wherein the antiallergic agent is thromboxane A2 receptor antagonist or leukotriene C4/D4 antagonist.

(8) The method according to (1), wherein the steroid is cortisteroid.

(9) The method according to (6), wherein the H1 blocker is Diphenhydramine.

(10) The method according, to (6), wherein the H2 blocker is Ranitidine.

(11) The method according to (7), wherein the thromboxane A2 receptor antagonist is Ramatroban or Montelukast.

(12) The method according to (8), wherein the cortisteroid is Dexamethason or Hydrocortisone.

(13) A method for treating cancer which comprises administering to a patient in need thereof a therapeutically effective amount of a humanized antibody or a human antibody which specifically binds to ganglioside GD3 or the antibody fragment thereof,

wherein a therapeutically effective amount of at least two compounds selected from antihistamic agent, antiallergic agent and steroid are administered, before administering a therapeutically effective amount of the humanized antibody or the human antibody which specifically binds to ganglioside GD3 or the antibody fragment thereof.

(14) A method for suppressing infusion reaction which is caused by administering to a patient in need thereof a therapeutically effective amount of a humanized antibody or a human antibody which specifically binds to ganglioside GD3 or the antibody fragment thereof which comprises; administering to a patient in need thereof a therapeutically effective amount of at least two compounds selected from antihistamic agent, antiallergic agent and steroid,

before administering to the patient in need thereof a therapeutically effective amount of a humanized antibody or a human antibody which specifically binds to ganglioside GD3 or the antibody fragment thereof.

Cancer in the present invention is not limited so long as ganglioside GD3 is expressed on surface of the cancer. Examples of the cancer include solid cancer. Preferably, the solid cancer includes melanoma, glioma, neuroblastoma and the like.

A humanized antibody or a human antibody defined in the present invention is not limited so long as the antibody specifically binds to ganglioside GD3.

Examples of a humanized antibody include human chimeric antibodies and human complementary determining region (hereinafter refered to as “CDR”)-grafted antibody described below.

A human chimeric antibody is an antibody comprising an antibody H chain V region (hereinafter also referred to as “VH”) and an antibody L chain V region (hereinafter also referred to as “VL”) of a non-human animal, a human antibody H chain C region (hereinafter also referred to as “CH”) and a human antibody L chain C region (hereinafter also referred to as “CL”). The non-human animal may be any of mouse, rat, hamster, rabbit and the like, so long as a hybridoma can be prepared therefrom.

The human chimeric antibody used in the present invention can be produced by obtaining cDNAs encoding VH and VL from a hybridoma which produces a monoclonal antibody, inserting the cDNAs into an animal cell expression vector having genes encoding a human antibody CH and a human antibody CL to construct a human chimeric antibody expression vector, and introducing the vector into an animal cell to express the antibody.

Any CH of a human chimeric antibody may be used, so long as it belongs to human immunoglobulin (hereinafter referred to as “hIg”), but those of hIgG class are preferred, and any one of subclasses further belonging to hIgG, such as hIgG1, hIgG2, hIgG3 and hIgG4, can be used. Also, any CL of a human chimeric antibody may be used, so long as it belongs to hIg, and those of κ class or λ class can be used.

A human CDR-grafted antibody is an antibody in which CDR amino acid sequences of VH and VL of an antibody derived from a non-human animal are grafted into appropriate positions of VH and VL of an human antibody.

The human CDR-grafted antibody of the present invention can be produced by grafting CDR sequences of VH and VL of an antibody of a non-human animal into CDR sequences of VH and VL of an optional human antibody to construct cDNAs encoding V regions obtained, inserting the cDNAs into an animal cell expression vector having genes encoding human antibody CH and human antibody CL to construct a human CDR-grafted antibody expression vector, and then introducing the expression vector into an animal cell to express the antibody.

Any CH of human CDR-grafted antibody may be used, so long as it belongs to hIg, but those of hIgG class are preferred and any one of subclasses further belonging to hIgG, such as hIgG1, hIgG2, hIgG3 and hIgG4, can be used. Also, any CL of human CDR-grafted antibody may be used so long as it belongs to hIg, and those of κ class or λ class can be used.

Originally, a human antibody is an antibody naturally existing in the human body, but it also includes antibodies obtained from a human antibody phage library and a human antibody producing transgenic animal, prepared based on the recent advance in genetic engineering, cell engineering and developmental engineering techniques.

The antibody existing in the human body can be obtained, for example, by isolating a human peripheral blood lymphocyte, immortalizing it by infection with EB virus or the like, followed by cloning, culturing a lymphocyte which produces the antibody, and purifying the antibody from the culture mixture.

The human antibody library is a library in which an antibody fragment, such as Fab, scFv or the like, is expressed on the surface of a phage by inserting an antibody gene prepared from human B cell into the phage gene. A phage which expresses an antibody fragment having desired antigen binding activity can be recovered from the library by using the binding activity to an antigen-immobilized substrate as the index. The antibody fragment can be further converted into a human antibody molecule comprising two complete H chains and two complete L chains by genetic engineering techniques.

A human antibody-producing transgenic animal is an animal in which a human antibody gene is introduced into its cell. Specifically, a human antibody-producing transgenic animal can be produced by introducing a human antibody gene into a mouse ES cell, grafting the ES cell into an initial stage embryo of other mouse, and developing an animal. The human antibody may be produced and accumulated by obtaining a hybridoma producing a human antibody according to a hybridoma preparation method usually carried out in non human mammals and then culturing the hybridoma to obtain the human antibody in a culture mixture.

The antibody which specifically reacts with ganglyoside GD3 used in the method of the present invention is not limited so long as it specifically reacts with ganglioside GD3.

An example of the humanized antibody or human antibody used in the method of the present invention includes the antibody which comprises CDR1, CDR2 and CDR3 of the H chain V region having the amino acid sequences represented by SEQ ID NOs:5, 6 and 7, respectively; and CDR1, CDR2 and CDR3 of the L chain V region having the amino acid sequences represented by SEQ ID NOs:8, 9 and 10, respectively.

An example of the CDR-grafted antibody used in the method of the present invention includes the antibody which comprises the H chain V region of the antibody having the amino acid sequence represented by SEQ ID NO:1 and the L chain V region of the antibody having the amino acid sequence represented by SEQ ID NO:2.

An example of the human chimeric antibody used in the method of the present invention includes the antibody which comprises the H chain V region of the antibody having the amino acid sequence represented by SEQ ID NO:3 and the L chain V region of the antibody comprises the amino acid sequence represented by SEQ ID NO:4.

Specific examples of the antibody used in the method of the present invention include human chimeric antibodies disclosed in U.S. Pat. No. 5,750,078, U.S. Pat. No. 5,866,692, U.S. Pat. No. 5,807,548, U.S. Pat. No. 6,437,098, U.S. Pat. No. 6,495,666, US2003-0166876, US2003-0095964, WO2001/23432, WO2002/78739, EP1,384,487 and EP 1,238,985, CDR-grafted antibodies disclosed in WO2001/23432, EP 1,238,985, WO2002/78739, EP1,384,487 or the like.

An antibody fragment defined in the present invention is not limited so long as the antibody fragment comprises a part of Fc region or Fc region.

The antibody fragment includes Fab, Fab′, F(ab′)₂, scFv, dsFv, a peptide comprising CDR and the like.

An Fab is an antibody fragment having a molecular weight of approximately 50,000 and an antigen binding activity, in which about a half of the N-terminal side of H chain and a full L chain, among fragments obtained by treating IgG with a protease, such as papain which cut at the amino acid residue at position 224 in the H chain, are bound together through a disulfide bond.

The Fab of the present invention can be obtained by treating an antibody which specifically reacts with ganglioside GD3 with a protease, papain. Also, the Fab can be obtained by inserting a DNA encoding Fab of the antibody into an expression vector, and introducing the vector into a host cell to express the Fab.

An F (ab′)₂ is an antibody fragment having a molecular weight of approximately 100,000 and an antigen binding activity, which is slightly larger than the Fab bound via a disulfide bond of the hinge region, among fragments obtained by treating IgG with a protease, pepsin (cut at the amino acid residue at position 234 in the H chain).

The F(ab′)₂ of the present invention can be obtained by treating an antibody which specifically reacts with ganglioside GD3, with a protease, pepsin. Also, the F(ab′)₂ can be obtained by binding Fab′ described below via a thioether bond or a disulfide bond.

An Fab′ is an antibody fragment having a molecular weight of approximately 50,000 and an antigen binding activity, which is obtained by cutting a disulfide bond of the hinge region of the F(ab′)₂.

The Fab′ of the present invention can be obtained by treating F(ab′)₂ which specifically reacts with ganglioside GD3 with a reducing agent, dithiothreitol. Also, the Fab′ can be produced by inserting DNA encoding an Fab′ fragment of the antibody into an expression vector, and introducing the vector into a host cell to express the Fab′.

An scFv is a VH-P-VL or VL-P-VH polypeptide in which one chain VH and one chain VL are linked using an appropriate peptide linker (hereinafter referred to as “P”). The VH and VL in the scFv used in the present invention may be derived from any one of antibodies obtained by a hybridoma, a humanized antibody and a human antibody.

The scFv of the present invention can be produced by obtaining cDNAs encoding the VH and VL of an antibody which specifically reacts with ganglioside GD3, constructing DNA encoding scFv, inserting the DNA into an expression vector, and then introducing the expression vector into a host cell to express the scFv.

A dsFv is obtained by binding polypeptides in which one amino acid residue of each of VH and VL is substituted with a cysteine residue via a disulfide bond between the cysteine residues. The amino acid residue substituted with a cysteine residue can be selected based on a three-dimensional structure estimation of the antibody in accordance with the method shown by Reiter et al. [Protein Engineering, 7, 697 (1994)]. The VH and VL in the dsFv of the present invention may be derived from any one of antibodies obtained by a hybridoma, a humanized antibody and a human antibody.

The dsFv of the present invention can be obtained by obtaining cDNAs encoding the VH and VL of an antibody which specifically reacts with ganglioside GD3, constructing DNA encoding dsFv, inserting the DNA into an expression vector, and then introducing the expression vector into a host cell to express the dsFv.

A peptide comprising CDR is constituted by at least one CDR in H chain and L chain. Plural CDRs can be bound directly or via an appropriate peptide linker.

The peptide comprising CDR of the present invention can be obtained by obtaining cDNA encoding the VH and VL of an antibody which specifically reacts with ganglioside GD3, inserting the cDNA into an expression vector, and then introducing the expression vector into a host cell to express the peptide comprising CDR.

Also, the peptide comprising CDR can be obtained by a chemical synthesis method such as Fmoc method (fluorenylmethoxycarbonyl method) or tBoc method (t-butyloxycarbonyl method).

The compounds used in the present invention can be any compounds so long as they are included in antihistamic agent, antiallergic agent and steroid. At least two compounds can be selected from same agents or different agents.

Antihistamic agent used in the method of the present invention includes H1 blocker, H2 blocker, or the like.

H1 blocker includes Fexofenadine HCl, Cetirizine HCl, Diphenhydramine HCl, Desloratradine, Hydroxyzine HCl, Promethazine HCl or the like. Fexofenadine HCl includes Allegra (Bland Name). Cetrizine HCl includes Zyrtec (Bland Name). Diphenhydramine HCl includes Benadryl (Bland Name). Desloratradine includes Clarinex (Bland Name). Hydroxyzine HCl includes Atarax (Brand Name) and Vistaril (Brand Name). Promethazine HCl includes Phenergan (Bland Name).

H2 blocker includes Cimetidine HCl, Famotidine, Ranitidine, Nizatidine, or the like. Cimetidine HCl includes Tagamet (Bland Name). Famotidine includes Pepcid (Bland Name). Ranitidine includes Zantac (Bland Name). Nizatidine includes Axid (Bland Name).

Antiallergic agent used in the method of the present invention includes thromboxane A2 receptor antagonist, leukotriene C4/D4 antagonist or the like. Thromboxane A2 receptor antagonist includes Ramatroban, Seratrodast or the like. Leukotriene C4/D4 antagonist includes Montelukast Zafirlukast or the like.

Ramatroban includes Baynas (Bland Name). Seratrodast includes BRONICA (Bland Name). Montelukast includes Singulair (Bland Name). Zafirlukast includes ACCOLATE (Bland Name).

Steroid used in the method of the present invention includes corticoidor the like. Corticoid includes Cortisone, Hydrocortisone, Hydrocortisone sodium phosphate, Hydrocortisone sodium succinate, Prednisolone, Prednisone, Methylprednisolone, Methylprednisolone sodium succinate, Triamcinolone, Triamcinolone acetonide, Paramethasone acetonide, Betamethasone, Dexamethason, Dexamethasone sodium phosphate or the like.

Cortisone includes Cortone (Bland Name). Hydrocortisone includes Hydrocortone (Bland Name), Cortril and the like. Hydrocortisone sodium phosphate includes Hydrocortone phosphate (Bland Name). Hydrocortisone sodium succinate includes Solu-Cortef (Bland Name). Prednisolone includes Prednisolone (Bland Name), Predonine (Bland Name) or the like. Methylprednisolone include DEPO-MEDROL (Bland Name), Medrol (Bland Name) or the like. Methylprednisolone sodium succinate includes Solu-Medrol (Bland Name). Triamcinolone includes Ledercort (Bland Name). Triamcinolone acetonide includes Kenacort-A (Bland Name). Paramethasone acetate includes Paramesone (Bland Name). Betamethasone includes CELESTONE (Bland Name). Dexamethason includes Decadron (Bland Name), Corson (Bland Name) or the like.

Dexamethasone sodium phosphate includes Decadron (Bland Name), Corson (Bland Name) Orgadrone (Bland Name) or the like.

An example of a combination of compounds selected from same agents includes a combination of a compound selected from H1 blockers and a compound selected from H2 blockers.

An example of a combination of a compound selected from H1 blockers and a compound selected from H2 blockers includes a combination of Benadryl (Bland name) and Zantac (Bland name).

An example of a combination of at least two compounds selected from different agents includes a combination of a compound selected from antihistamic agents and a compound selected from antiallergic agents, a combination of a compound selected from antihistamic agents and a compound selected from steroid, a combination of two compounds selected from antiallergic agents and a compound selected from steroid.

An example of a combination of a compound selected from antihistamic agents and a compound selected from antiallergic agents includes a combination of Singulair (Bland name) and Zyrtec (Bland name), a combination of Benadryl (Bland name), Zantac (Bland name), Singulair (Bland name) and Zyrtec (Bland name).

An example of a combination a compound selected from antihistamic agents and a compound selected from steroid includes Benadryl (Bland name), Zantac (Bland name) and Dexamethasone (Bland name).

An example of a combination of two compounds selected from antiallergic agents and a compound selected from steroid includes Benadryl (Bland name), Zantac (Bland name), Singulair (Bland name), Zyrtec (Bland name) and Dexamethasone (Bland name).

Pharmaceutical composition comprising, as an active ingredient, the antibody used in the method of the present invention may be solely administered to a patient in need thereof after administering to the patient a therapeutically effective amount of at least two compounds selected from antihistamic agent, antiallergic agent and steroid:

The compounds may be administered before the administration of the antibody, and it may further be administered simultaneously with the antibody or after the administration of the antibody.

Normally, pharmaceutical composition is preferably provided as pharmaceutical preparations which are produced by mixing with one or more pharmaceutical acceptable carriers according to any method known in the field of pharmaceutics.

It is preferable to use an administration which is most effective in carrying out a treatment. Examples include oral administration and parenteral administration such as intraoral, bronchial, intrarectal, subcutaneous, intramuscular, intravenous administrations and the like. In an antibody-containing pharmaceutical formulation, intravenous administration is preferrable.

Examples of dosage form include sprays, capsules, tablets, granules, syrups, emulsions, suppositories, injection, an ointments, tapes and the like.

Examples of formulation suitable for oral administration include emulsions, syrups, capsules, tablets, powders, granules and the like.

Liquid preparations, such as emulsions and syrups, can be produced by using as an additive, water; sugar, such as sucrose, sorbitol, fructose; glycol, such as polyethylene glycol, propylene glycol; oil, such as sesami oil, olive oil, soybean oil; antiseptic such as p-hydroxy benzoate ester; flavoring, such as strawberry flavors, peppermint flavors; and the like.

Capsules, tablets, powders, granules or the like can be produced by using as an additive, excipients such as lactose, glucose, sucrose, mannitol; disintegrators, such as starch, sodium alginate; lubricants, such as magnesium stearate, talc; binders, such as polyvinyl alcohol, hydroxypropylcellulose, gelatin; surfactants, such as fatty acid ester; plasticizers, such as glycerine; and the like.

Examples of pharmaceutical preparations suitable for parenteral administration include injectables, suppositories, sprays and the like.

An injection is prepared by using a carrier or the like which comprises a saline solution, a glucose solution, a mixture of both or the like.

A suppository is prepared by using a carrier, such as cacao butter, hydrogenated fat, carboxylic acid and the like.

A spray is prepared by using the antibody preparation itself or using a carrier or the like which facilitates absorption by allowing the compound to disperse as fine particles without stimulating the mouth cavity and bronchial mucous membrane of a recipient.

Examples of carriers include lactose, glycerine and the like. Preparations, such as aerosol and dry powder, can be used, depending on the properties of the antibody and the carrier to be used. In addition, these parenteral preparations can be supplemented with components illustrated as additives for oral preparations.

The applied dose and the number of administration vary depending on target therapeutic effects, administration methods, treatment period, age and body weight of the patient. Normally, 10 mg/m² to 50 mg/m², preferably 2 mg/m² to 40 mg/m², more preferably 40 mg/m² are one or more times administered per 1 week to 2 month, preferably 2 weeks to 1 month to an adult patient.

In the method of the present invention, each patient can be treated according to a premedication regimen with at least two compounds selected from antihistamic agent, antiallergic agent and steroid, immediately to 1 week before, preferably 6 hours to 1 day before, more preferably 3 to 12 hours before treatment with the antibody.

The premedication in the method of the present invention can be conducted one or more times, preferably three times.

Preferably, each patient can be treated first with antiallergic agent, antihistamic agent and steroid to one day before treatment with the antibody, second with antiallergic agent, antihistamic agent and steroid 6 hours before treatment with the antibody, and third with antihistamic agent and steroid immediately before treatment with the antibody.

In particular, each patient can be treated first with antihistamic agent such as Diphenhydramine and Ranitidine and steroid such as Dexamethasone to 1 day before treatment with the antibody, second with antiallergic agent such as Montelukast, antihistamic agent such as Certirizine, Diphenhydramine and Ranitidine and steroid such as Dexamethasone to 6 hours before treatment with the antibody, and third with Montelukast as antiallergic agent and Certirizine, Diphenhydramine and antihistamic agent such as Ranitidine immediately before treatment with the antibody.

As the dose to be used in the premedication, 1/20 to 2.5 times of the dose of the antibody, preferably 1/10 to 1.25 the dose of the antibody, more preferably 1/5 to 1 the dose of the antibody can be used. As the frequency of dosage can be every 14 days, a correspondingly lower incidence of infusion reactions is expected. If necessary, the dose can be decreased by one-half according to tolerance of the patient and at the discretion of the treating oncologist, although this is to be avoided as much as possible as it will constitute a protocol deviation.

The present invention relates to a method for suppressing infusion reaction which is caused by administering to a patient in need thereof a therapeutically effective amount of a humanized antibody or a human antibody which specifically binds to ganglioside GD3 or the antibody fragment thereof which comprises; administering to a patient in need thereof a therapeutically effective amount of at least two compounds selected from antihistamic agent, antiallergic agent and steroid, before administering to the patient in need thereof a therapeutically effective amount of a humanized antibody or a human antibody which specifically binds to ganglioside GD3 or the antibody fragment thereof.

Infusion reaction used in the present invention is a type of side effect.

An example of infusion reaction in the method of the present invention includes itching, fever, hives, chills and rash.

Processes for producing a humanized antibody against ganglioside GD3 are explained below.

1. Production of Humanized Antibody

(1) Construction of Humanized Antibody Expression Vector

The humanized antibody expression vector is an expression vector for animal cells to which genes encoding CH and CL of a human antibody are inserted, and can be constructed by cloning the genes encoding CH and CL of a human antibody into an expression vector for animal cells. The C regions of a human antibody can be any human antibody CH and CL, and examples include a C region belonging to IgG1 subclass in an H chain of a human antibody (hereinafter referred to as “hCγ1”) and a C region belonging to K class in an L chain of a human antibody (hereinafter referred to as “hCκ”). A chromosomal DNA comprising an exon and an intron can be used as the gene encoding CH and CL of a human antibody, and cDNA can also be used.

Any expression vector for animal cells can be used, so long as a gene encoding the human antibody C region can be inserted and expressed. Examples include pAGE107 [Cytotechnology, 3, 133 (1990)], pAGE103 [J. Biochem., 101, 1307 (1987)], pHSG274 [Gene, 27, 223 (1984)], PKCR [Proc. Natl. Acad. Sci. U.S.A., 78, 1527 (1981)], pSG1βd2-4 [Cytotechnology, 4, 173(1990)] and the like. The promoter and the enhancer used in the expression vector for animal cell include early promoter and enhancer of SV40 [J. Biochem., 101, 1307 (1987)], LTR promoter and enhancer of Moloney mouse leukemia virus [Biochem. Biophys. Res. Comun., 149, 960 (1987)], promoter [Cell, 41, 479 (1985)] and enhancer [Cell, 33, 717 (1983)] of immunoglobulin H chain, and the like.

The humanized antibody expression vector may be either of a type in which a gene encoding an antibody H chain and a gene encoding an antibody L chain exist on separate vectors or of a type in which both genes exist on the same vector (hereinafter referred to as “tandem type”). In view of easiness to construct a humanized antibody expression vector, easiness to introduce into animal cells, and balance between the expression levels of antibody H and L chains in animal cells, a tandem type of the humanized antibody expression vector is more preferred [J. Immunol. Methods, 167, 271 (1994)]. The tandem type humanized antibody expression vector includes pKANTEX93 (WO 97/10354), pEE18 [HYBRIDOMA, 17, 559 (1998)] and the like.

The thus constructed humanized antibody expression vector can be used for the expression of the human chimeric antibody and human CDR-grafted antibody in animal cells.

(2) Preparation of cDNA Encoding V Region of Antibody Derived from Non-Human Animal

cDNAs encoding VH and VL of an antibody derived from an non-human animal such as a mouse antibody are obtained as follows.

mRNA is extracted from hybridoma cells producing a mouse antibody or the like to synthesize cDNA. The synthesized cDNA is inserted into a vector such as a phage or a plasmid to prepare a cDNA library. Each of a recombinant phage or recombinant plasmid containing cDNA encoding VH and a recombinant phage or recombinant plasmid containing cDNA encoding VL is isolated from the library using a C region part or a V region part of a mouse antibody as the DNA probe.

The full nucleotide sequences of the VH and VL of the mouse antibody of interest on the recombinant phage or recombinant plasmid are determined, and the full amino acid sequences of the VH and VL are deduced from the nucleotide sequences.

The non-human animal may be any animal such as a mouse, a rat, a hamster or a rabbit, so long as a hybridoma cell can be produced therefrom.

The method for preparing total RNA from a hybridoma cell includes a guanidine thiocyanate-cesium trifluoroacetate method [Methods in Enzymol., 154, 3 (1987)] and the like. The method for preparing mRNA from total RNA includes an oligo (dT) immobilized cellulose column method [Molecular Cloning: A Laboratory Manual, Cold Spring Harbor Lab. Press, New York (1989), hereinafter referred to as “Molecular Cloning: A Laboratory Manual”] and the like. Also, a kit for preparing mRNA from a hybridoma cell includes Fast Track mRNA Isolation Kit (manufactured by Invitrogen), Quick Prep mRNA Purification Kit (manufactured by Pharmacia) and the like.

The method for synthesizing cDNA and preparing a cDNA library includes known methods (Molecular Cloning: A Laboratory Manual; Current Protocols in Molecular Biology, Supplement 1-34, hereinafter referred to as “Current Protocols in Molecular Biology”); a method using a commercially available kit such as Super Script™ Plasmid System for cDNA Synthesis and Plasmid Cloning (manufactured by GIBCO BRL) or ZAP-cDNA Kit (manufactured by Stratagene); and the like.

The vector into which the cDNA synthesized using mRNA extracted from a hybridoma cell as a template is inserted for preparing a cDNA library may be any vector, so long as the cDNA can be inserted. Examples include ZAP Express [Strategies, 5, 58 (1992)], pBluescript II SK(+) [Nucleic Acids Research, 17, 9494 (1989)], λzapII (manufactured by Stratagene), λgt10 and λgt11 [DNA Cloning: A Practical Approach, I, 49 (1985)], Lambda BlueMid (manufactured by Clontech), λExCell and pT7T3 18U (manufactured by Pharmacia), pcD2 [Mol. Cell. Biol., 3, 280 (1983)], pUC18 [Gene, 33, 103 (1985)] and the like.

Any E. coli for introducing the cDNA library constructed by a phage or plasmid vector can be used, so long as the cDNA library can be introduced, expressed and maintained. Examples include XL1-Blue MRF′ [Strategies, 5, 81 (1992)], C600 [Genetics, 39, 440 (1954)], Y1088 and Y1090 [Science, 222, 778 (1983)], NM522 [J. Mol. Biol., 166, 1 (1983)], K802 [J. Mol. Biol., 16; 118 (1966)], JM105 [Gene, 38, 275 (1985)] and the like.

For selecting cDNA clones encoding VH and VL of an antibody derived from a non-human animal in the cDNA library, a colony hybridization or plaque hybridization method using an isotope- or fluorescence-labeled probe can be used (Molecular Cloning: A Laboratory Manual). Also, the cDNAs encoding VH and VL can be prepared through polymerase chain reaction (hereinafter referred to as “PCR”; Molecular Cloning: A Laboratory Manual; Current Protocols in Molecular Biology) by preparing primers and using cDNA prepared from mRNA or a cDNA library as the template.

The nucleotide sequence of the cDNA can be determined by digesting the cDNA selected according to the above method with appropriate restriction enzymes and the like, cloning the fragments into a plasmid such as pBluescript SK(−) (manufactured by Stratagene), carrying out the reaction according to a usually used nucleotide analyzing method such as the dideoxy method of Sanger, F. et al. [Proc. Natl. Acad. Sci. USA, 74, 5463 (1977)], and then analyzing the sequence using an automatic nucleotide sequence analyzer such as A.L.F. DNA sequencer (manufactured by Pharmacia).

Whether the obtained cDNAs encode the full amino acid sequences of the VH and VL of the antibody containing a secretory signal sequence can be confirmed by estimating the full amino acid sequences of the VH and VL from the determined nucleotide sequence and comparing them with full amino acid sequences of the VH and VL of known antibodies [Sequences of Proteins of Immunological Interest, US Dept. Health and Human Services (1991), hereinafter referred to as “Sequences of Proteins of Immunological Interest”].

(3) Analysis of Amino Acid Sequence of V Region Derived from a Non-Human Animal

Regarding the complete amino acid sequences of VH and VL of the antibody comprising a secretory signal sequence, the length of the secretory signal sequence and N-terminal amino acid sequences can be deduced and subgroups to which they belong can also be deduced, by comparing with full amino acid sequences of VH and VL of known antibodies (Sequences of Proteins of Immunological Interest). Also the amino acid sequence of each CDR of VH and VL can be deduced by comparing it with amino acid sequences of VH and VL of known antibodies (Sequences of Proteins of Immunological Interest).

(4) Construction of Human Chimeric Antibody Expression Vector

A human chimeric antibody expression vector can be constructed by cloning cDNAs encoding VH and VL of an antibody derived from a non-human animal in the region upstream of genes encoding CH and CL of the human antibody on the humanized antibody expression vector as described in the item 1(1). For example, each of cDNAs encoding VH and VL of an antibody derived from a non-human animal is linked with a synthesized DNA comprising nucleotide sequences at the 3′ terminals of VH and VL of an antibody derived from an on-human animal and nucleotide sequences at the 5′ terminals of CH and CL of a human antibody and having a recognition sequence of an appropriate restriction enzyme at both terminals. Each cDNA is cloned so that it is appropriately expressed in upstream of genes encoding the CH and CL of the humanized antibody expression vector as described in the item 1(1) to thereby construct a human chimeric antibody expression vector.

(5) Construction of cDNA Encoding V Region of Human CDR-Grafted Antibody

cDNAs encoding VH and VL of a human CDR-grafted antibody can be obtained as follows. First, amino acid sequences of FRs in VH and VL of a human antibody to which amino acid sequences of CDRs in VH and VL of an antibody derived from a non-human animal antibody are grafted are selected. Any amino acid sequences of FRs in VH and VL of a human antibody can be used, so long as they are derived from human. Examples include amino acid sequences of FRs in VH and VL of human antibodies registered in database such as Protein Data Bank, amino acid sequences common to subgroups of FRs in the VH and VL of human antibodies (Sequences of Proteins of Immunological Interest) and the like. In order to produce a human CDR-grafted antibody having potent activity, amino acid sequences having high homology (at least 60% or more) with amino acid sequence of FRs of VH and VL of an antibody of interest derived from a non-human animal is preferably selected. Then, amino acid sequences of CDRs of VH and VL of the antibody derived from a non-human animal are grafted to the selected amino acid sequences of FRs of VH and VL of a human antibody to design amino acid sequences of the VH and VL of a human CDR-grafted antibody. The designed amino acid sequences are converted to DNA sequences by considering the frequency of codon usage found in nucleotide sequences of genes of antibodies (Sequence of Proteins of Immunological Interest), and the DNA sequences encoding the amino acid sequences of the VH and VL of a human CDR-grafted antibody are designed. Several synthetic DNAs having a length of about 100 nucleotides are synthesized, and PCR is carried out using them. In this case, it is preferred that 6 synthetic DNAs in each of the H chain and the L chain are designed in view of the reaction efficiency of PCR and the lengths of DNAs which can be synthesized.

Furthermore, they can be easily cloned into the humanized antibody expression vector constructed in the item 1(1) by introducing the recognition sequence of an appropriate restriction enzyme to the 5′ terminal of the synthetic DNAs present on the both terminals. After the PCR, an amplified product is cloned into a plasmid such as pBluescript SK (−) (manufactured by Stratagene), and the nucleotide sequences are determined according to the method described in the item 1(2) to obtain a plasmid having DNA sequences encoding the VH and VL of a designed human CDR-grafted antibody.

(6) Modification of Amino Acid Sequence of V Region of Human CDR-Grafted Antibody

It is known that when a human CDR-grafted antibody is produced by simply grafting CDRs in VH and VL of an antibody derived from a non-human animal in FRs of the VH and VL of a human antibody, its antigen binding activity is reduced in comparison with that of the original antibody derived from a non-human animal [BIO/TECHNOLOGY, 9, 266 (1991)]. As the reason, it is considered that several amino acid residues in not only CDRs but also FRs directly or indirectly relate to the antigen binding activity in the VH and VL of the original antibody derived from a non-human animal, and that these amino acid residues are changed to different amino acid residues derived from FRs of the VH and VL of the human antibody. In order to resolve the problem, in human CDR-grafted antibodies, attempts have been made to identify, among amino acid sequences of the FR of the VH and VL of human antibodies, an amino acid residue which directly relates to binding to the antigen, an amino acid residue which is interacted with an amino acid residue of CDR or an amino acid residue which keeps three-dimensional structure of the antibody and directly relates to its binding to the antigen, and to increase the reduced antigen binding activity by changing them into amino acid residues of the original antibody derived from a non-human animal [BIO/TECHNOLOGY, 9, 266 (1991)]. In producing a human CDR-grafted antibody, it is the most important point to efficiently identify these FR amino acid residues related to the antigen binding activity, so that construction and analysis of the three-dimensional structure of antibodies have been carried out by X-ray crystallography [J. Mol. Biol., 112, 535 (1977)], computer-modeling [Protein Engineering, 7, 1501 (1994)] and the like. Although the information of the three-dimensional structure of antibodies has been useful in the production of a human CDR-grafted antibody, no method for producing a human CDR-grafted antibody which can be applied to any antibodies has been established yet. Therefore, various attempts must currently be necessary, for example, several modified antibodies of each antibody are produced and the relationship between each of the modified antibodies and its antibody binding activity is examined.

Substitution of the amino acid residues of the FR in the VH and VL of a human antibody can be achieved by carrying out the PCR described in the item 1(5) using synthetic DNA for modification. With regard to an amplified product obtained by the PCR, the nucleotide sequence is determined according to the method described in the item 1(2) so that whether the objective modification has been carried out is confirmed.

(7) Construction of Human CDR-Grafted Antibody Expression Vector

A human CDR-grafted antibody expression vector can be constructed by cloning cDNAs encoding VH and VL of the human CDR-grafted antibody constructed in the items 1(5) and 1(6) into upstream of the genes encoding CH and CL of the human antibody in the humanized antibody expression vector as described in the item 1(1).

For example, when recognition sites for an appropriate restriction enzymes are introduced to the 5′-terminal of synthetic DNAs positioned at both terminals among synthetic DNAs used in the construction of VH and VL of the human CDR-grafted antibody in the items 1(5) and 1(6), a human CDR-grafted antibody expression vector can be constructed by carrying out cloning so that they are expressed in an appropriate form in upstream of genes encoding CH and CL of the human antibody in the humanized antibody expression vector as described in the item 1(1).

(8) Transient Expression of Humanized Antibody

In order to efficiently evaluate the antigen binding activity of various humanized antibodies produced, the humanized antibodies can be expressed transiently using the humanized antibody expression Vector as described in the items 1(4) and 1(7) or the modified expression vector thereof. Any cell can be used as a host cell, so long as the host cell can express a humanized antibody. Generally, COS-7 cell (ATCC CRL1651) is used in view of its high expression level [Methods in Nucleic Acids Res., CRC Press, 283 (1991)]. The method for introducing the expression vector into COS-7 cell includes a DEAE-dextran method [Methods in Nucleic Acids Res., CRC Press, 283 (1991)], a lipofection method [Proc. Natl. Acad. Sci. USA, 84, 7413 (1987)], and the like.

After introduction of the expression vector, the expression level and antigen binding activity of the humanized antibody in the culture supernatant can be measured by enzyme-linked immunosorbent assay [hereinafter referred to as “ELISA”; Antibodies: A Laboratory Manual, Cold Spring Harbor Laboratory, Chapter 14 (1988), hereinafter referred to as “Antibodies: A Laboratory Manual”, Monoclonal Antibodies: Principles and Practice, Academic Press Limited (1996), hereinafter referred to as “Monoclonal Antibodies” (1996)] and the like.

(9) Stable Expression of Humanized Antibody

A transformant which produces a humanized antibody stably can be obtained by introducing into an appropriate host cell the humanized antibody expression vector described in the items 1(4) and 1(7).

The method for introducing the expression vector into a host cell includes electroporation [Japanese Published Unexamined Patent Application No. 257891/90, Cytotechnology, 3, 133 (1990)] and the like.

Any cell can be used as the host cell into which the humanized antibody expression vector is to be introduced, so long as it can express a humanized antibody. Examples include mouse SP2/0-Ag14 cell (ATCC CRL1581), mouse P3X63-Ag8.653 cell (ATCC CRL1580), CHO cell in which a dihydrofolate reductase gene (hereinafter referred to as “dhfr”) is defective [Proc. Natl. Acad. Sci. U.S.A., 77, 4216 (1980)], rat YB2/3HL.P2.G11.16Ag.20 cell (ATCC CRL1662, hereinafter referred to as “YB2/0 cell”), and the like.

A host cell which expresses an antibody having high ADCC activity includes host cells in which activity of an enzyme protein relating to synthesis of an intracellular sugar nucleotide, GDP-fucose, an enzyme protein relating to modification of a sugar chain in which 1-position of fucose is bound to 6-position of N-acetylglucosamine in the reducing end through a-bond in a complex N-glycoside-linked sugar chain, or a protein relating to transport of an intracellular sugar nucleotide, GDP-fucose to the Golgi body or the like is decreased or deleted, preferably YB2/0 cell, and the like.

After introduction of the expression vector, a transformant which expresses a humanized antibody stably is selected, by culturing it in a medium for animal cell culture containing an agent such as G418 sulfate (hereinafter referred to as “G418”, manufactured by SIGMA) in accordance with the method disclosed in Japanese Published Unexamined Patent Application No. 257891/90. The medium for animal cell culture includes PRMI1640 medium (manufactured by Nissui Pharmaceutical), GIT medium (manufactured by Nihon Pharmaceutical), EX-CELL302 medium (manufactured by JRH), IMDM medium (manufactured by GIBCO BRL), Hybridoma-SFM medium (manufactured by GIBCO BRL), media obtained by adding various additives such as fetal bovine serum (hereinafter referred to as “FBS”) to these media, and the like. The resulting transformant is cultured in a medium to thereby form and accumulate the humanized antibody in the culture supernatant. The expression level and antigen binding activity of the humanized antibody in the culture supernatant can be measured by ELISA or the like. Also, in the transformant, the expression level of the humanized antibody can be increased by using the dhfr amplification system or the like according to the method disclosed in Japanese Published Unexamined Patent Application No. 257891/90.

The humanized antibody can be purified from the culture supernatant of the transformant by using a protein A column [Antibodies, A Laboratory Manual, Monoclonal Antibodies (1996)]. Any other conventional methods for protein purification can be used. For example, the humanized antibody can be purified by a combination of gel filtration, ion-exchange chromatography, ultrafiltration and the like. The molecular weight of the H chain or the L chain of the purified humanized antibody or the antibody molecule as a whole is determined by polyacrylamide gel electrophoresis [hereinafter referred to as “SDS-PAGE”, Nature, 227, 680 (1970)], Western blotting [Antibodies: A Laboratory Manual, Monoclonal Antibodies (1996)], and the like.

(10) Evaluation of Activity of Humanized Antibody

The binding activity to an antigen and the binding activity to a cultured cancer cell line of the purified humanized antibody can be measured by ELISA, an immunofluorescent method [Cancer Immunol. Immunother., 36, 373 (1993)] and the like. The cytotoxic activity against an antigen positive culture cell line can be evaluated by measuring the CDC activity, the ADCC activity or the like [Cancer Immunol. Immunother., 36, 373 (1993)].

The present invention will be described below based on Examples, but the present invention is not limited thereto.

### EXAMPLE 1

### Premedication by a Combination of Various Compounds for Suppressing Infusion Reaction Caused by the Administration of Human Anti-GD3 Chimeric Antibody (1)

The following test was performed to suppress the infusion reactions caused by human anti-GD3 chimeric antibody.

A human chimeric antibody which specifically binds to ganglioside GD3, wherein the human chimeric antibody comprises the H chain V region of the antibody comprising the amino acid sequence represented by SEQ ID NO:3 and the L chain V region of the antibody comprising the amino acid sequence represented by SEQ ID NO:4 was produced according to U.S. Pat. No. 6,437,098 (hereinafter referred to as ‘KM-871’).

KM-871 was administered to each patient with a dose of 40 mg/m² once every two weeks for four times as a general rule. Benadryl (50 mg) and Zantac (50 mg) were parenterally administered to the patient 30 minutes prior to each administration of KM-871 except for the first administration.

As a test dose, KM-871 (10 mg/m²), was administered to the patient two weeks prior to the first administration of KM-871 to confirm its safeness. When toxicity or side effects were observed to the patient by the administration, the administration was withdrawn.

The results are shown in Table 1.

TABLE 1

| Patient No. | Test | 1st administration | 2nd administration | 3rd administration | 4th administration | Pre-medication |
|---|---|---|---|---|---|---|
| 1 | | ITCHING | | | | 2nd |
| 2 | FEVER | ARMS, LEGS, ERYTHEMA | CHILLS | | withdrawn | 1st |
| 3 | ITCHING | FULLNESS IN THROAT | HIVES | | | 1st and 2nd |
| 4 | HIVES | FEVER | FEVER | HIVES | withdrawn | 1st |
| 5 | | RIGORS | | CHILLS | withdrawn | 2nd |
| 6 | | RIGORS | | | | 1st |

By the premedication of Benadryl and Zantac, 5 cases out of 6 cases showed the subsidence of symptoms such as Itching, Chills, Rigors and the like.

As mentioned above, premedication by a combination of Benadryl and Zantac suppressed infusion reactions such as Urticaria, Itching, Rigors and the like more effectively compared to the premedication by Benadryl alone.

### EXAMPLE 2

### Premedication by a Combination of Various Compounds for Suppressing Infusion Reactions Caused by the Administration of KM-871 (2)

The following test was performed by using various compounds to suppress the infusion reactions caused by KM-871.

KM-871 was administered to each patient with a dose of 40 mg/m² once every two weeks for four times as a general rule. Sigulair (10 mg) and Zertec (10 mg) were orally administered to the patient 30 minutes prior to each administration of KM-871 except for the first administration.

As a test dose, KM-871 (10 mg/m²), was administered to the patient two weeks prior to the first administration of KM-871 to confirm its safeness. When toxicity or infusion reactions were observed to the patient by the administration, the administration was withdrawn.

The results are shown in Table 2.

TABLE 2

| Patient No. | Test | 1st administration | 2nd administration | 3rd administration | 4th administration | Pre-medication |
|---|---|---|---|---|---|---|
| 1 | FACIAL FLUSHING | ITCHING | | | withdrawn | 2nd and 3rd |
| 2 | | HIVES | | | withdrawn | 2nd and 3rd |
| 3 | | CHILLS | | | withdrawn | 2nd and 3rd |
| 4 | | THROAT SWELLING | | | withdrawn | 2nd and 3rd |
| 5 | | ITCHING/HIVES | | | | 3rd and 4th |
| 6 | HIVES/ITCHING | CHILLS | HIVES | HIVES ON HANDS | | 1st |

By the premedication of Sigulair and Zertec, 5 cases out of 6 cases showed the subsidence of symptoms such as Itching, HIVES and the like.

### EXAMPLE 3

### Premedication by a Combination of Various Compounds for Suppressing Infusion Reactions Caused by the Administration of KM-871 (3)

The following test was performed by using various compounds to suppress the infusion reactions caused by KM-871.

KM-871 was administered to each patient with a dose of 40 mg/m² once every two weeks for four times as a general rule. Sigulair (10 mg) and Zertec (10 mg) were orally administered and Benadryl (50 mg) and Zantac (50 mg) were parenterally administered to the patient 30 minutes prior to each administration of KM-871 except for the first administration.

As a test dose, KM-871 (10 mg/m²), was administered to the patient two weeks prior to the first administration of KM-871 to confirm its safeness. When toxicity or infusion reactions were observed to the patient by the administration, the administration was withdrawn.

The results are shown in Table 3.

TABLE 3

| Patient No. | Test | 1st administration | 2nd administration | 3rd administration | 4th administration | Pre-medication |
|---|---|---|---|---|---|---|
| 1 | | HIVES | | | withdrawn | 1st, 2nd and 3rd |
| 2 | | ITCHING | | | withdrawn | 1st, 2nd and 3rd |
| 3 | | FEVER | ITCHING | | | 2nd and 4th |
| 4 | | HIVES/ITCHING | | | | 2nd and 4th |
| 5 | | FEVER | | | | 2nd, 3rd and 4th |

By the premedication of Benadryl, Zantac, Sigulair and Zertec, all cases showed the subsidence of symptoms such as Fever, Itching, HIVES and the like.

### EXAMPLE 4

### Premedication by a Combination of Various Compounds for Suppressing Infusion Reactions Caused by the Administration of KM-871 (4)

The following test was performed by using various compounds to suppress the infusion reactions caused by KM-871.

KM-871 was administered to each patient with a dose of 40 mg/m² or 80 mg/m² every two weeks for four times as a general rule. Sigulair (10 mg) and Zertec (10 mg) were orally administered and Benadryl (50 mg), Zantac (50 mg) and Dexametazone (20 mg) were parenterally administered to the patient 30 minutes prior to each administration of KM-871 except for the first administration.

As a test dose, KM-871 (10 mg/m²), was administered to the patient two weeks prior to the first administration of KM-871 to confirm its safeness. When toxicity or infusion reactions are observed to the patient by the administration, the administration was withdrawn.

The results are shown in Table 4.

| Patient No. | Test | 1st administration | 2nd administration | 3rd administration | 4th administration | Pre-medication |
|---|---|---|---|---|---|---|
| 1 | RASH | HIVES | | | withdrawn | 2nd |

By the premedication of Benadryl, Zantac, Sigulair and Zertec, all cases showed the subsidence of symptoms such as rash, HIVES and the like.

### EXAMPLE 5

### Premedication by a Combination of Various Compounds for Suppressing Infusion Reactions Caused by the Administration of KM-871 (5)

In the first administration, Rash, Urticaria, Itching had appeared to the patient who had been administered 4 mg/m² of KM-871. On the day of the second administration of KM-871, Benadryl (100 mg), Zantac (50 mg), Singulair (10 mg), Zyrtec (10 mg) and Dexametazone (10 mg) were administered to the patient followed by the administration of KM-871.

As a result, infusion reactions did not appear after the second administration. Prior to the third administration of KM-871, Benadryl (100 mg), Zantac (50 mg), Singulair (10 mg), and Zyrtec (10 mg), were administered to the patient.

As a result, infusion reactions did not appear after the third administration. The fourth administration was withdrawn because the tumor of an affected area of the patient in this test example had swollen.

### EXAMPLE 6

### Premedication by a Combination of Various Compounds for Suppressing Infusion Reactions Caused by the Administration of KM-871 (6)

KM-871 can be administered by i.v. infusion into a peripheral vein or via central venous access. An infusion route can be secured in advance and infusion of physiologic saline solution for injection can be initiated. A specified premedication regimen can be administered as follows:

At bedtime the day before the infusion:

dexamethasone 8 mg p.o.

diphenhydramine 25 mg p.o.

ranitidine 25 mg p.o.

the morning of the infusion:

dexamethasone 8 mg p.o.

diphenhydramine 50 mg p.o.

Singulair 10 mg p.o.

immediately prior to the infusion:

dexamethasone 10 mg i.v.

diphenhydramine 25 mg i.v.

ranitidine 50 mg i.v.

If any patient experiences any grade 2 (Common Terminology Criteria for Adverse Events (CTCAE) v3.0) infusion reaction, the infusion must be interrupted, along with administration of a bolus of 200 mg hydrocortisone and 50 mg diphenhydramine, and 10 mg Singulair p.o.

The infusion can be restarted once the symptoms have subsided. If the symptoms do not subside, or if the reaction recurs immediately on rechallenge, or if the patient experiences any infusion-related adverse reaction higher than grade 2, the patient is to be withdrawn. In addition, 8 mg dexamethasone p.o. can be administered 6 hours following completion of the KM-871 infusion.

Premedication Regimen

| | | | Immediately Prior to |
|---|---|---|---|
| | At Bedtime | In the morning | Infusion |
| Dexamethasone | 8 mg p.o | 8 mg p.o. | 10 mg i.v. |
| Diphenhydramine | 25 mg p.o. | 50 mg p.o. | 25 mg i.v. |
| Ranitidine | 25 mg p.o. | NONE | 50 mg i.v. |
| Singulair | NONE | 10 mg p.o. | NONE |

Infusion Interruptions Regimen

| | For Infusion |
|---|---|
| | Interruptions |
| Hydrocortisone | 200 mg bolus i.v. |
| Diphenhydramine | 50 mg bolus i.v. |
| Singulair | 10 mg p.o. |

Post-medication Regimen

| | 6 hours Post-infusion |
|---|---|
| Dexamethasone | 8 mg p.o. |
| Diphenhydramine | PRN¹⁾ for infusion reaction |
| Ranitidine | NONE |
| Singulair | PRN¹⁾ for infusion reaction |

¹⁾pro re nata

What is the claims:

1. A method for treating cancer which comprises;
administering to a patient in need thereof a therapeutically effective amount of at least two compounds selected from antihistamic agent, antiallergic agent and steroid,
and then administering to the patient in need thereof a therapeutically effective amount of a humanized antibody or a human antibody which specifically binds to ganglioside GD3 or the antibody fragment thereof.

2. The method according to claim 1, wherein the humanized antibody is a human chimeric antibody or a human complementary determining region (CDR)-grafted antibody.

3. The method according to claim 1, wherein the humanized antibody or human antibody comprises:
CDR1, CDR2 and CDR3 of the H chain V region having the amino acid sequences represented by SEQ ID NOs: 5, 6 and 7, respectively; and
CDR1, CDR2 and CDR3 of the L chain V region having the amino acid sequences represented by SEQ ID NOs: 8, 9 and 10, respectively.

4. The method according to claim 2, wherein the CDR-grafted antibody comprises:
the H chain V region of the antibody comprising the amino acid sequence represented by SEQ ID NO:1; and
the L chain V region of the antibody comprising the amino acid sequence represented by SEQ ID NO:2.

5. The method according to claim 2, wherein the human chimeric antibody comprises:
the H chain V region of the antibody comprising the amino acid sequence represented by SEQ ID NO:3; and
the L chain V region of the antibody comprising the amino acid sequence represented by SEQ ID NO:4.

6. The method according to claim 1, wherein the antihistamic agent is H1 blocker or H2 blocker.

7. The method according to claim 1, wherein the antiallergic agent is thromboxane A2 receptor antagonist or leukotriene C4/D4 antagonist.

8. The method according to claim 1, wherein the steroid is cortisteroid.

9. The method according to claim 6, wherein the H1 blocker is Diphenhydramine.

10. The method according to claim 6, wherein the H2 blocker is Ranitidine.

11. The method according to claim 7, wherein the thromboxane A2 receptor antagonist is Ramatroban or Montelukast.

12. The method according to claim 8, wherein the cortisteroid is Dexamethason or Hydrocortisone.

13. A method for treating cancer which comprises administering to a patient in need thereof a therapeutically effective amount of a humanized antibody or a human antibody which specifically binds to ganglioside GD3 or the antibody fragment thereof,
wherein a therapeutically effective amount of at least two compounds selected from antihistamic agent, antiallergic agent and steroid are administered, before administering a therapeutically effective amount of the humanized antibody or the human antibody which specifically binds to ganglioside GD3 or the antibody fragment thereof.

14. A method for suppressing infusion reaction which is caused by administering to a patient in need thereof a therapeutically effective amount of a humanized antibody or a human antibody which specifically binds to ganglioside GD3 or the antibody fragment thereof which comprises; administering to a patient in need thereof a therapeutically effective amount of at least two compounds selected from antihistamic agent, antiallergic agent and steroid,
before administering to the patient in need thereof a therapeutically effective amount of a humanized antibody or a human antibody which specifically binds to ganglioside GD3 or the antibody fragment thereof.


SEQUENCE LISTING (10 sequences)

SEQ ID NO: 1 - protein, 119 aa, Mus musculus
EVTLVESGGDFVKPGGSLKVSCAASGFAFSHYAMSWVRQTPAKRLEWVAYISSGGSGTYYSDSVKGRFTISRDNAKNTLYLQMRSLRSEDSAMYFCTRVKLGTYYFDSWGQGTTLTVSS

SEQ ID NO: 2 - protein, 108 aa, Mus musculus
DIQMTQTASSLPASLGDRVTISCSASQDISNYLNWYQQKPDGTVKLLIFYSSNLHSGVPSRFSGGGSGTDYSLTISNLEPEDIATYFCHQYSKLPWTFGGGTKLEIKR

SEQ ID NO: 3 - protein, 119 aa, Artificial Sequence
  Artificial Sequence H chain V region of the antibody
EVQLVESGGDFVQPGGSLRVSCAASGFAFSHYAMSWVRQAPGKGLEWVAYISSGGSGTYYSDSVKGRFTISRDNSKNTLYLQMRSLRAEDSAVYFCTRVKLGTYYFDSWGQGTLLTVSS

SEQ ID NO: 4 - protein, 108 aa, Artificial Sequence
  Artificial Sequence L chain V region of the antibody
DIQMTQSPSSLSASVGDRVTITCSASQDISNYLNWYQQKPDKAPKLLIFYSSNLHSGVPSRFSGGGSGTDYTLTISSLQPEDIATYFCHQYSKLPWTFGQGTKVEIKR

SEQ ID NO: 5 - protein, 5 aa, Mus musculus
HYAMS

SEQ ID NO: 6 - protein, 17 aa, Mus musculus
YISSGGSGTYYSDSVKG

SEQ ID NO: 7 - protein, 10 aa, Mus musculus
VKLGTYYFDS

SEQ ID NO: 8 - protein, 11 aa, Mus musculus
SASQDISNYLN

SEQ ID NO: 9 - protein, 7 aa, Mus musculus
YSSNLHS

SEQ ID NO: 10 - protein, 9 aa, Mus musculus
HQYSKLPWT